(19) United States
(12) Patent Application Publication
(10) Pub. No.: US 2014/0296694 A1
(43) Pub. Date: Oct. 2, 2014

(54) METHOD AND SYSTEM FOR ULTRASOUND NEEDLE GUIDANCE

(71) Applicant: General Electric Company, (US)

(72) Inventor: William J. Jaworski, Wauwatosa, WI (US)

(73) Assignee: General Electric Company, Schenectady, NY (US)

(21) Appl. No.: 13/855,488

(22) Filed: Apr. 2, 2013

(51) Int. Cl. A61B 5/06; A61B 8/08

(52) U.S. Cl. CPC A61B 5/066; A61B 8/0841
USPC 600/409; 600/439; 600/424

(57) ABSTRACT

A method and medical system for providing needle guidance. The method and system includes acquiring ultrasound data while manipulating a needle and tracking the needle tip while manipulating the needle. The method and system includes displaying a first live image including at least a portion of the needle in a first viewing pane and displaying a second live image including the needle tip in a second viewing pane at the same time as the first live image. The second live image includes a portion of the first live image at a greater level of zoom than the first live image.

### FIELD OF THE INVENTION

This disclosure relates generally to a method and system for tracking a position of a needle tip and displaying a zoomed-in image of the needle tip at the same time as an overview image.

### BACKGROUND OF THE INVENTION

During an interventional ultrasound procedure, a clinician is constantly concerned about the location and trajectory of a needle inserted into a patient. The clinician needs to clearly understand exactly where the needle tip is located for both patient safety and clinical effectiveness. In order to complete a successful interventional procedure, the clinician must accurately position the needle tip in the desired anatomy while avoiding causing any undue tissue damage during the process of inserting and positioning the needle. In addition to avoiding particular anatomical regions, oftentimes the clinician is trying to position the needle in extremely close proximity to other structures. In order to safely accomplish an interventional ultrasound procedure, the clinician needs to accurately comprehend the full path of the needle as well as the position of the needle tip with respect to specific anatomy.

In order to easily understand the path of the needle, it is desirable to view an overview image showing the needle and the surrounding anatomy. An overview image helps provide context to the clinician regarding the real-time location of the needle with respect to the patient's anatomy. However, in order to most effectively understand the position of the needle tip, it is desirable to view an image of the needle tip with an increased level of zoom compared to the overview image. Using an image of the needle tip with a higher level of zoom allows the clinician to confidently position the needle tip in exactly the desired location with respect to the patient's anatomy. Due to the higher level of zoom, any movement of the needle will be amplified in the zoomed-in view. Therefore, if the clinician inserts or moves the needle significantly, the needle tip will no longer be visible in the zoomed-in view. If a zoomed-in view of the needle tip is desired with a conventional system, the clinician must manually select a region-of-interest that includes the needle tip. At high levels of zoom, it is necessary for the clinician to constantly adjust the position of the region-of-interest. This is both inconvenient and time-consuming for the clinician. Additionally, in some cases, the lack of detailed information regarding the needle tip location could be potentially dangerous for the patient.

For these and other reasons an improved method and medical system for needle guidance is desired.

### BRIEF DESCRIPTION OF THE INVENTION

The above-mentioned shortcomings, disadvantages and problems are addressed herein which will be understood by reading and understanding the following specification.

In an embodiment, a method of needle guidance includes acquiring ultrasound data during the process of manipulating a needle in a patient, tracking a needle tip of the needle during the process of manipulating the needle in the patient, and displaying a first live image including at least a portion of the needle in a first viewing pane based on the ultrasound data. The method includes displaying a second live image including the needle tip in a second viewing pane at the same time first live image. The second live image includes a portion of the first live image at a greater level of zoom than the first live image.

In another embodiment, a method of ultrasound needle guidance includes acquiring ultrasound data of a first region-of-interest including a needle and displaying a first live image in a first viewing pane, where the first live image includes an overview image defined by the first region-of-interest. The method includes tracking a position of a needle tip as the needle is inserted and establishing a second region-of-interest around the needle tip. The method includes automatically adjusting a position of the second region-of-interest to track with the needle tip as the needle is inserted. The method includes displaying a second live image defined by the second region-of-interest in a second viewing pane at the same time as the first live image. The second live image includes the needle tip at a greater level of zoom than the first live image.

In another embodiment, a medical system for providing needle guidance includes a needle including a needle tip, a probe including a plurality of transducer elements, a display device, and a processor. The processor is configured to control the probe to acquire ultrasound data from a first region-of-interest and track the needle tip while the needle is moved. The processor is configured to define a second region-of-interest including a subset of the first region-of-interest and to adjust a position of the second region-of-interest to track with the needle tip while the needle is moved. The processor is configured to display a first live image of the first region-of-interest on the display device based on the ultrasound data and to display a second live image of the second region-of-interest on the display device at the same time as the first live image. The second live image includes the needle tip and is at a greater level of zoom than the first live image.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of a medical system in accordance with an embodiment;

FIG. 2 is a flow chart of a method in accordance with an embodiment;

FIG. 3 is a schematic representation of a display format in accordance with an embodiment; and

FIG. 4 is a schematic representation of a display format in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

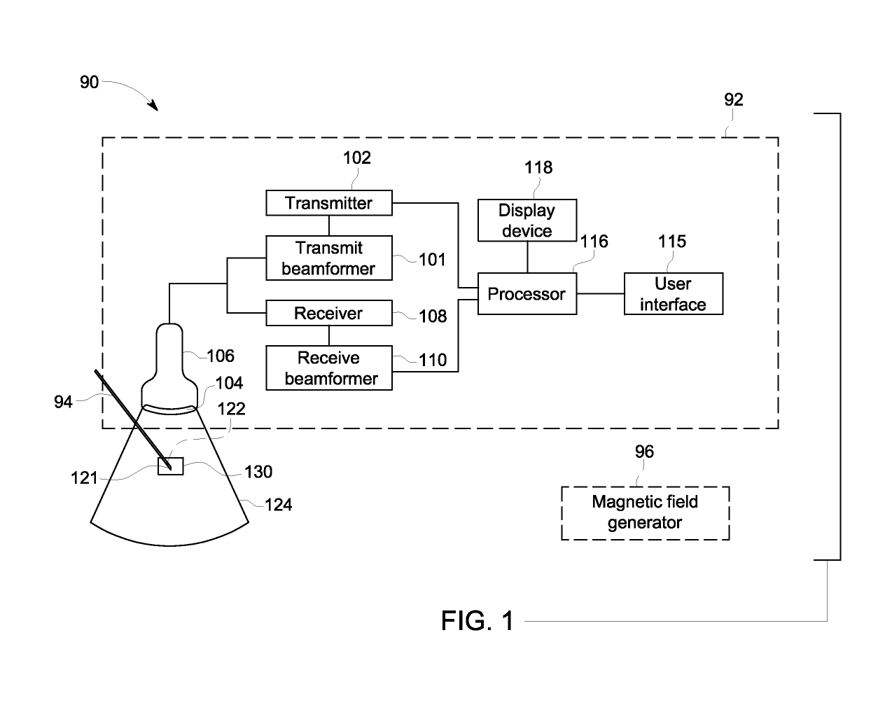

FIG. 1 is a schematic diagram of a medical system 90 in accordance with an embodiment. The medical system 90 includes an ultrasound imaging system 92, a needle, 94, and, optionally, a magnetic field generator 96. The ultrasound imaging system 92 includes a transmit beamformer 101 and a transmitter 102 that drive transducer elements 104 within a probe 106 to emit pulsed ultrasonic signals into a patient (not shown). A variety of geometries of ultrasound probes and transducer elements 104 may be used. The pulsed ultrasonic signals are back-scattered from structures in the patient, like blood cells or muscular tissue, to produce echoes that return to the transducer elements 104. The echoes are converted into electrical signals, or ultrasound data, by the transducer elements 104 in the probe 106 and the electrical signals are received by a receiver 108. According to other embodiments, the probe 106 may contain electronic circuitry to do all or part of the transmit beamforming and/or the receive beamforming. For example, all or part of the transmit beamformer 101, the transmitter 102, the receiver 108 and the receive beamformer 110 may be disposed within the probe 106 according to other embodiments. The terms “scan” or “scanning” may also be used in this disclosure to refer to acquiring ultrasound data through the process of transmitting and receiving ultrasonic signals. For purposes of this disclosure, the term “ultrasound data” may include data that was acquired or processed by an ultrasound system. Additionally, the term “data” may also be used in this disclosure to refer to either one or more datasets. The electrical signals representing the received echoes are passed through the receive beamformer 110 that outputs ultrasound data. A user interface 115 may be used to control operation of the ultrasound imaging system 92. The user interface 115 may include one or more controls such as a keyboard, a rotary, a mouse, a trackball, a track pad, and a touch screen. The user interface 115 may, for example, be used to control the input of patient data, to change a scanning parameter, or to change a display parameter.

The ultrasound imaging system 92 also includes a processor 116 in electronic communication with the probe 106. The processor 116 may control the transmit beamformer 101, the transmitter 102 and, therefore, the ultrasound beams emitted by the transducer elements 104 in the probe 106. The processor 116 may also process the ultrasound data into images for display on a display device 118. According to an embodiment, the processor 116 may also include a complex demodulator (not shown) that demodulates the RF ultrasound data and generates raw ultrasound data. The processor 116 may be adapted to perform one or more processing operations on the ultrasound data according to a plurality of selectable ultrasound modalities. The ultrasound data may be processed in real-time during a scanning session as the echo signals are received. For the purposes of this disclosure, the term “real-time” is defined to include a process that is performed without any intentional delay, such as process that is performed with less than a 300 mS delay. Additionally or alternatively, the ultrasound data may be stored temporarily in a buffer (not shown) during a scanning session and processed in less than real-time in a live or off-line operation. Some embodiments may include multiple processors (not shown) to handle the processing tasks. For example, a first processor may be utilized to demodulate and decimate the RF signal while a second processor may be used to further process the data prior to displaying an image. It should be appreciated that other embodiments may use a different arrangement of processors to handle the processing tasks.

The ultrasound imaging system 92 may continuously acquire ultrasound data at a frame rate of, for example, 10 Hz to 30 Hz. Images generated from the ultrasound data may be refreshed at a similar frame rate. Other embodiments may acquire and display ultrasound data at different rates. For example, some embodiments may acquire ultrasound data at a frame rate of less than 10 Hz or greater than 30 Hz depending on the parameters used for the data acquisition. A memory (not shown) may be included for storing processed frames of acquired ultrasound data. The memory should be of sufficient capacity to store at least several seconds of ultrasound data. The memory may include any known data storage medium.

Optionally, embodiments of the present invention may be implemented utilizing contrast agents. Contrast imaging generates enhanced images of anatomical structures and blood flow in a body when using ultrasound contrast agents such as microbubbles. After acquiring ultrasound data while using a contrast agent, the image analysis includes separating harmonic and linear components, enhancing the harmonic component, and generating an ultrasound image by utilizing the enhanced harmonic component. Separation of harmonic components from the received signals is performed using suitable filters. The use of contrast agents for ultrasound imaging is well-known by those skilled in the art and will therefore not be described in further detail.

In various embodiments of the present invention, ultrasound data may be processed by different mode-related modules (e.g., B-mode, Color Doppler, M-mode, Color M-mode, spectral Doppler, TVI, strain, strain rate, and the like) to form 2D or 3D image frames. The frames are stored and timing information indicating the time when the data was acquired in memory may be recorded. The modules may include, for example, a scan conversion module to perform scan conversion operations to convert the image frames from coordinate beam space to display space coordinates. A video processor module may be provided that reads the image frames from a memory and displays the image frames in real-time while a procedure is being carried out on a patient. A video processor module may store the image frames in an image memory, from which the images are read and displayed.

The medical system 90 may also include magnetic field generator 96, and the needle may include an electromagnetic sensor 122 according to an embodiment. The magnetic field generator 96 may comprise one or more sets of coils adapted to generate an electromagnetic field. The processor 116 is in communication with the electromagnetic sensor 122. According to an embodiment, the electromagnetic sensor 122 may include three sets of coils, where each set of coils is disposed orthogonally to the two other sets of coils. For example, a first set of coils may be disposed along an x-axis, a second set may be disposed along a y-axis, and a third set may be disposed along a z-axis. Different currents are induced in each of the three orthogonal coils by the electromagnetic field from the magnetic field generator 96. By detecting the currents induced in each of the coils, position and orientation information may be determined for the electromagnetic sensor 122. The processor 116 is able to determine the position and orientation of the probe 106 based on the data from the electromagnetic sensor 122. Using a field generator and an electromagnetic sensor to track the position and orientation of a device within a magnetic field is well-known by those skilled in the art and, therefore, will not be described in additional detail. While the embodiment of FIG. 1 uses a field generator and an electromagnetic sensor, it should be appreciated by those skilled in the art that other embodiments may use other methods and sensor types for obtaining position and orientation information of the needle 94. For example, embodiments may use an optical tracking system, including a system where multiple light-emitting diodes (LEDs) or reflectors are attached to the needle 94, and a system of cameras is used to determine the position of the LEDs or reflectors through triangulation or other methods.

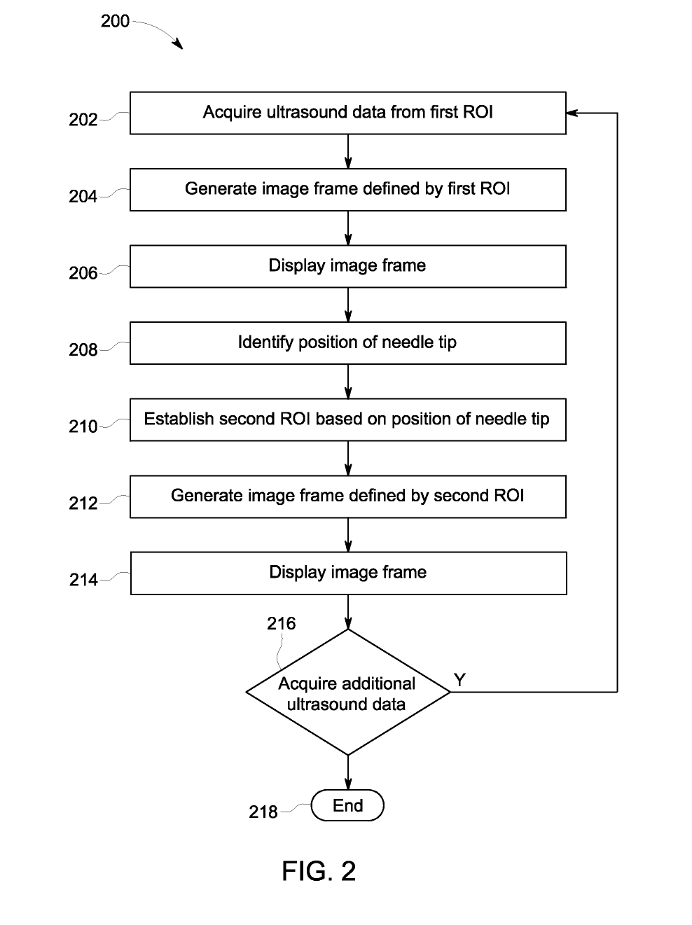

FIG. 2 is a flow chart of a method 200 in accordance with an embodiment. The individual blocks represent steps that may be performed in accordance with the method 200. Additional embodiments may perform the steps shown in a different sequence and/or additional embodiments may include additional steps not shown in FIG. 2. The technical effect of the method 200 is the tracking of a needle tip and the display of a zoomed-in image of the needle tip as a needle is inserted.

According to an exemplary embodiment, the method 200 may be performed with the medical system 90. Referring to both FIGS. 1 and 2, at step 202 the processor 116 controls the transmitter 102, the transmit beamformer 101, the probe 106, the receiver 108, and the receive beamformer 110 to acquire ultrasound data from a first region-of-interest 124, hereinafter first ROI 124. For purposes of this disclosure, the term ROI may be defined to include the region from which ultrasound data is acquired. The size and shape of the first ROI 124 may be selected by the user through the user interface 115 or the first ROI 124 may be the size of a field-of-view of the probe 106 in a particular setting. The processor 116 may control the ultrasound imaging system 92 to acquire one or more frames of data from the first ROI 124 at step 202. At step 204, the processor 116 generates an image frame based on ultrasound data acquired from the first ROI 124. At step 206, the processor 116 displays the image frame generated at step 204 on the display device 118. The display of the image frame at step 206 will be described in additional detail hereinafter.

Next, at step 208, the processor 116 identifies the position of the needle tip 121. According to an exemplary embodiment, the processor 116 may implement an image processing technique to identify a representation of the needle tip 121 in the image frame generated at step 204. For example, the processor 116 may apply a template-matching algorithm in order to identify the position of the needle tip 121 in the image frame.

According to an exemplary embodiment, the processor 116 may use a template, or mask, shaped like the needle tip. The template-matching algorithm may search the entire image frame for a region with the highest correlation to the template. The processor 116 may, in effect, slide the template across the image frame while searching for the region with the highest correlation. Since the needle 94 and needle tip 121 may be at any orientation in the image frame, the processor 116 may additionally compare the template to various regions of the image frame with the template in a number of different rotational positions. According to an embodiment, the processor 116 may rotate the template through all possible rotations for each template-sized region of the image frame. The processor 116 may, for example, calculate differences in pixel intensities between the template and the image from for all the possible positions and rotations of the template in the image frame. The processor 116 may then sum the differences of all the pixels for each template position/orientation in order to generate a correlation coefficient. The processor 116 may identify the position of the needle tip 121 by identifying the position and orientation of the template on the image that yields the highest correlation coefficient. According to other embodiments, the template and the image frame may both be down-sampled prior to performing the template-matching in order to decrease the computational load on the processor 116. According to yet other embodiments, the template-matching may be performed in a frequency domain after performing a Fourier analysis of the image frame. Template-matching is an example of one image processing technique that could be used to identify the position of the needle tip 94. It should be appreciated that any other image processing technique may be used to identify the position of the needle tip 121 according to other embodiments.

According to other embodiments, non-image processing techniques may be used to identify the position of the needle tip 121. For example, referring to FIG. 1, the needle 94 may include the optional electromagnetic sensor 122, and the medical system 90 may include the magnetic field generator 96. The electromagnetic sensor 122 may be either attached to the needle 94 or the needle 94 may be manufactured with the electromagnetic sensor 122 as an integrated component. According to an embodiment, the magnetic field generator 96 generates a magnetic field with known physical properties. For example, the magnetic field may have specified gradients in the x-direction, the y-direction, and the z-direction. The electromagnetic sensor 122 may include three coils, each coil disposed in a mutually orthogonal position. Each coil in the electromagnetic sensor 122 is adapted to detect the magnetic field in a specific orientation with respect to the needle 94. By analyzing the signals from the coils of the electromagnetic sensor 122, the processor 116 may calculate the position and orientation of the needle 94 and, therefore, the needle tip 121 with respect to the magnetic field generated by the magnetic field generator 96. According to an embodiment, the processor 116 may utilize a look-up table including dimensions for a large number of needles or other interventional devices. The look-up table may, for example, contain precise information regarding the location of the needle tip 121 with respect to the electromagnetic sensor 122. By tracking the position of the electromagnetic sensor 122 with respect to the magnetic field, the processor 116 is able to track the position of the needle tip 121 in real-time.

According to another embodiment, other types of tracking systems may be used to identify the position of the needle tip 121. For example, an optical tracking system may be used to identify the position of the needle tip 121. An optical tracking system may, for example, include a stationary array of cameras and multiple light-emitting diodes (LEDs) or reflectors attached to the needle 94. The LEDs or reflectors may be attached to end of the needle 94 opposite of the needle tip 121. The LEDs or reflectors are intended to remain outside of the patient, where they may be detected by the array of cameras. The processor 116 may detect the LEDs or reflectors based on the images captured by the array of cameras. Based on the size and orientation of the LEDs or reflectors, the processor 116 may calculate the position and orientation of the needle 94. It should be appreciated that the techniques described hereinabove for identifying the position of the needle tip 121 represent just a subset of the possible techniques that may be used to identify the position of the needle tip 121. Additional embodiments may use any other technique to determine the position of the needle tip 121.

Referring to FIGS. 1 and 2, at step 210, the processor 116 establishes a second ROI based on the position of the needle tip. An exemplary second ROI 130 is shown in FIG. 1. The second ROI 130 is positioned to include the needle tip 121 and the second ROI 130 represents just a subset of the first ROI 124. According to an embodiment, the processor 116 uses the position of the needle tip 121 that was identified during step 208 in order to establish the second ROI 130. The size of the second ROI 130 may be predetermined, or the size of the second ROI 130 may be user-configurable. However, it is important that the size of the second ROI 130 is smaller than the size of the first ROI 124. According to an embodiment, the processor 116 may position the second ROI 130 so that the needle tip 121 is positioned in the center of the second ROI 130. The second ROI 130 is shown as rectangular in shape in FIG. 1. However, it should be appreciated that the second ROI may be any other shape, including circular or oval.

Next, at step 212, the processor 116 generates an image frame defined by the second ROI 130. Then, at step 214, the image frame generated at step 212 is displayed. The image frame defined by the second ROI 130 may be based on the ultrasound data acquired at step 202. According to another embodiment, the method 200 may be modified to include an additional step in between steps 210 and 212. Specifically, the processor may acquire additional ultrasound data specifically from the second ROI. Then, the image frame generated at step 212 may be based on the additional ultrasound data acquired from the second ROI 130. Additional information about the display of the image frame defined by the second ROI 130 will be discussed hereinafter.

At step 216, the method 200 returns to step 202 if it is desired to acquire additional ultrasound data. As long as additional ultrasound data is desired, the method 200 iteratively repeats steps 202, 204, 206, 208, 210, 212, 214, and 216. According to an embodiment, additional image frames are generated at steps 204 and 212 each time enough ultrasound data is acquired to generate an additional frame. Each iteration of steps 202, 204, 206, 208, 210, 212, 214, and 216 results in the display of an updated image frame generated based on ultrasound data from the first ROI and the display of an updated image frame based on ultrasound data from the second ROI. Each updated image frame is displayed in a manner so that it replaces the previously displayed image frame from the corresponding ROI as part of a live ultrasound image. Multiple iterations of the method 200 result in live images comprising a series of image frames acquired from the same ROI at different points in time. There are many factors that influence the frame rate of a live ultrasound image including the size of the ROI and the type of acquisition, but frames rates in the range of 10 to 60 frames per second would be within the expected range. It should be appreciated by those skilled in the art that frames of ultrasound data may be acquired at either a faster rate or a slower rate according to other embodiments. Each repetition through steps 202, 204, 206, 208, 210, 212, 214, and 216 results in the generation and display of an additional image frame representing the first ROI and an additional image frame representing the second ROI. Additionally, each iteration of steps 202, 204, 206, 208, 210, 212, 214, and 216 results in an updated identification of the position of the needle tip 121 at step 208. By repeatedly identifying the position of the needle tip 121, the method 200 effectively tracks the position of the needle tip 121 Likewise, the processor 116 establishes the position of the second ROI based on the most recently identified needle tip position. The processor 116 may reposition the second ROI so that the position of the second ROI tracks the motion of the needle tip. According to many embodiments, ultrasound data will be acquired more-or-less constantly during the multiple iterations of the method 200. The live images are updated each time enough data has been acquired to generate an additional image frame. According to an embodiment, both a first live image defined by the first ROI and a second live image defined by the second ROI are displayed at the same time. The second ROI is a subset of the first ROI in an exemplary embodiment. Therefore, the second live image may be generated based on a subset of the ultrasound data used to generate the first live image. Or, the second live image may be generated based on an acquisition of ultrasound data limited to the second ROI. That is, the first live image and the second live image may be based on ultrasound data from separate acquisitions. Live images are well-known to those skilled in the art and will, therefore, not be described in additional detail. If it is not desired to acquire additional ultrasound data at step 216, the method 200 advances to step 218 and ends.

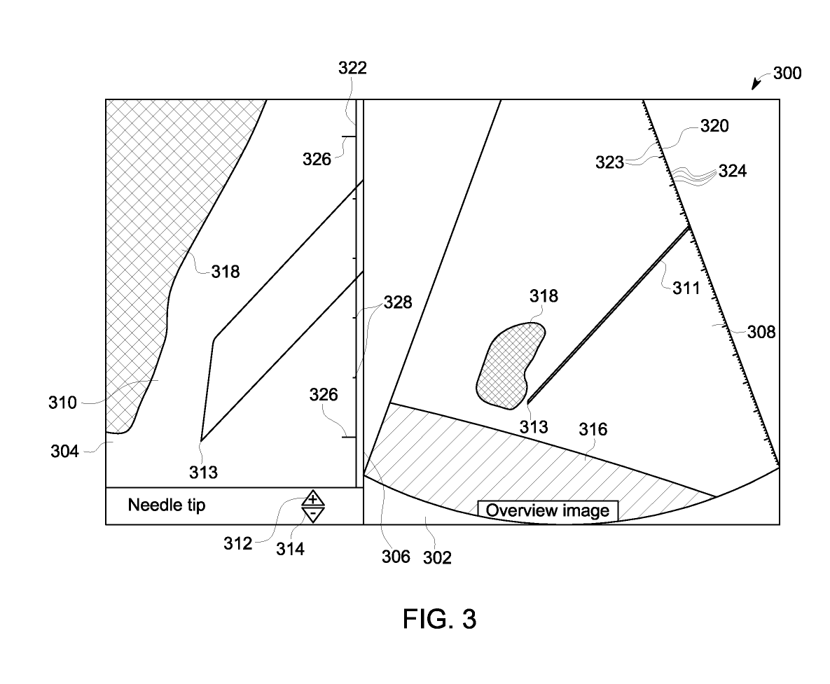

FIG. 3 is schematic representation of a display format in accordance with an embodiment. The display format 300 includes a first viewing pane 302 and a second viewing pane 304. The first viewing pane 302 is separated from the second viewing pane 304 by a divider 306 according to an embodiment. The display format 300 represents an exemplary output from a method such as the method 200. A first live image 308 is displayed in the first viewing pane 302 and a second live image 310 is displayed in the second viewing pane 304. The first live image 308 may comprise a sequence of image frames generated based on ultrasound data acquired from the first ROI 124 Likewise, the second live image 310 may comprise a sequence of image frames that are generated based on ultrasound data acquired from the second ROI 130. Or, as previously discussed, the second live image 310 may be a zoomed-in view of a portion of the first live image 308. Referring to FIGS. 1, 2 and 3, the first live image 308 may be defined by the first ROI 124, while the second live image 310 may be defined by the second ROI 130.

The display format 300 may optionally include a graphical user interface including one or more controls for adjusting a level of zoom in the second live image 310. For example, a graphical user interface including a zoom-in control 312 and a zoom-out control 314 is depicted in the display format 300. The first live image 308 and the second live image 310 are both updated as additional ultrasound data is acquired. Therefore, both the first live image 308 and the second live image 310 will accurately represent the real-time position of the needle 94 and the needle tip 121 as the needle 94 is being inserted or manipulated.

Additionally, the first live image 308 includes representations of a needle 311 and surrounding structures. A needle tip 313 is shown in the first live image 308 as well as a structure 316 and a structure 318. The first live image 308 provides an overview image and allows the clinician to easily understand the position of the needle 311 and the needle tip 313 with respect to the patient's anatomy. For example, the clinician may be trying to insert the needle 311 into structure 316. However, it may be critical for patient safety that structure 318 is not pierced by the needle 311. While the first live image 308 grants the clinician an excellent overview of the needle position, it does not allow the patient to see the needle tip 313 with a high degree of precision.

The second live image 310 provides the clinician with a zoomed-in view of just the needle tip 313 and the anatomy in close proximity to the needle tip 313. The second live image 310 represents the needle tip 313 at a higher level of zoom than the first live image 308. The second live image thus provides the clinician with a magnified view of the needle tip 313 in real-time. For example, structure 318 is shown with respect to the needle tip 313. Both the structure 318 and the needle tip 313 are magnified with respect to the first live image 308. Additionally, as described with respect to the method 200 (shown in FIG. 2), the processor 116 may update the position of the second ROI 130 (shown in FIG. 1) with the acquisition of each updated image frame. The method 200 adjusts the position of the second ROI 130 so that the second ROI 130 includes the needle tip 313 even as the needle 311 is being moved. As a result, the method 200 automatically tracks the needle tip 313, and the second live image 310 shows a real-time image of the needle tip 313 at a greater level of zoom than the overview image represented by the second live image 308. The second live image 310 provides the clinician with a detailed view of the needle tip 313 and the anatomy around the needle tip 313. By viewing both the first live image 308 and the second live image 310, the clinician is able to insert the needle 311 more efficiently and with a higher level of patient safety. The clinician may use the first live image 308 to provide a more global perspective of the position of the needle 311 while the needle is inserted. The clinician may also use the second live image 310 to more precisely position the needle tip 313. Since the second ROI tracks the needle tip, and since the second live image 310 represents the second ROI, the second live image automatically includes the needle tip 313 and the surrounding anatomy even as the position of the needle 311 is adjusted. The second live image provides a needle tip view that updates in real-time as the position of the needle tip 313 is adjusted. The second live image provides feedback allowing the clinician to safely position the needle tip 313 in exactly the desired position while avoiding sensitive structures within the patient. A first scale 320 is displayed with the first live image 308 in the first viewing pane 302, and a second scale 322 is displayed with the second live image 310 in the second viewing pane 304. The first scale 320 includes both major marks 323 and minor marks 324. Likewise, the second scale includes major marks 326 and minor marks 328. Since the second live image 310 has a higher level of zoom, the spacing of major and minor marks is greater on the second scale 322 than on the first scale 320. The second scale 322 allows the clinician to easily gauge the distance of the needle tip 313 from any relevant anatomy, such as the structure 318. Additionally, the clinician is able to easily determine the level of zoom in the second live image 310 by comparing the spacing of the major and minor marks between the first scale 320 and the second scale 322.

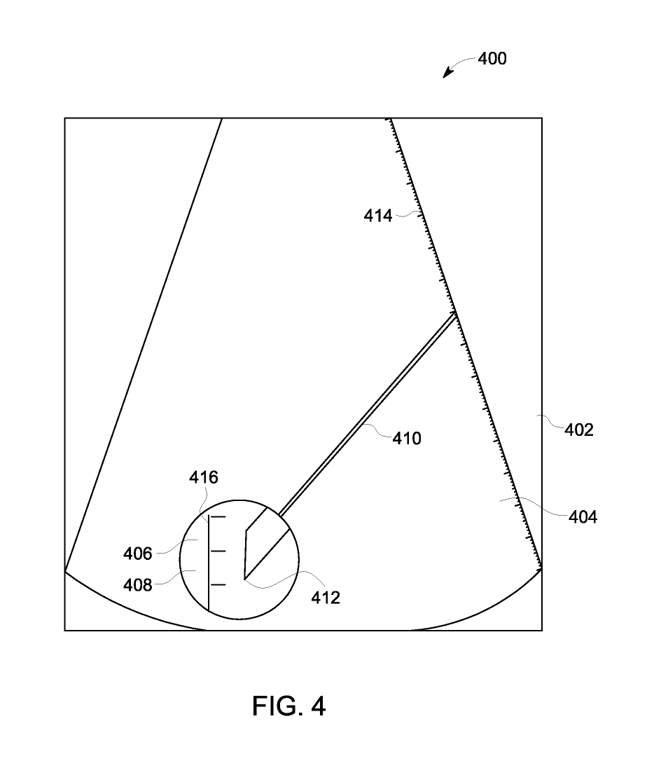

FIG. 4 is a schematic representation of a display format 400 in accordance with an embodiment. The display format 400 includes a first viewing pane 402 for displaying a first live image 404 and a second viewing pane 406 for displaying a second live image 408. According to an embodiment, the first live image 404 includes a needle 410. The second live image 408 includes magnified view of a needle tip 412 of the needle 410. The second live image 408 may be a magnified view of the first live image 404, or the second live image 408 may be generated based on separately acquired ultrasound data. For example, the second live image 408 may be generated based on ultrasound data that is specifically acquired from a smaller ROI than the ROI used to generate the first live image 404. According to an exemplary embodiment, the first live image 404 and the second live image 408 may be generated according to the method 200 shown in FIG. 2. As previously described, according to the method 200, the second ROI tracks the position of the needle tip 412 as the needle 410 is inserted. The second live image 408, therefore, includes the needle tip 412 even as the needle 410 is repositioned. According to the embodiment shown in FIG. 4, the second image pane 406 is positioned over the location where the needle tip would be positioned in the first viewing pane 402. The second live image 408 displayed in the second viewing pane 406, therefore, provides the effect of magnifying the needle tip 412. Since the second image pane 406 is superimposed on the location of the needle tip in the first live image 404, the second live image 408 obscures a portion of the first live image 404. However, the second live image 408 is of the needle tip 412 at a higher level of zoom than the first live image 404. A first scale 414 is displayed on the first live image 404, and a second scale 416 is displayed on the second live image 408. The clinician may use the first scale 414 and the second scale 416 to gauge both distances to relevant anatomical structures as well as the relative level of zoom between the first live image 404 and the second live image 408. According to an embodiment, the location of the second viewing pane 406 may move as the needle 410 is inserted. The processor 116 may control the position of the second image pane 406 in such a way so that the second viewing pane 406 moves in synchronization with the needle 410 as the needle 410 is moved. For example, the processor 116 may position the second viewing pane 406 so that it is positioned on top of the location where the needle tip would be in the first live image 404. The second image pane 406 may be centered on the location where the needle tip would be on the first live image 404 or the second viewing pane 406 may stay in place as long as the needle tip 412 remains viewable within the second viewing pane 406. According to an embodiment, the second viewing pane 406 may move only when the needle tip 412 is about to pass through the extent of the second viewing pane 406. According to an embodiment, the processor 116 may shift the second viewing pane 406 in the same direction that the needle tip 412 is moving relative to the first viewing pane 402.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

We claim:

1. A method of ultrasound needle guidance, the method comprising:
acquiring ultrasound data during the process of manipulating a needle in a patient;
tracking a needle tip of the needle during the process of manipulating the needle in the patient;
displaying a first live image including at least a portion of the needle in a first viewing pane based on the ultrasound data; and
displaying a second live image including the needle tip in a second viewing pane at the same time as the first live image, the second live image comprising a portion of the first live image at a greater level of zoom than the first live image.

2. The method of claim 1, wherein a portion of the ultrasound data used to generate the second live image is automatically selected to include the needle tip based on said tracking the needle tip.

3. The method of claim 1, wherein said tracking the needle tip comprises implementing an image processing technique to identify the needle tip in the first live image.

4. The method of claim 3, wherein said implementing the image processing technique comprises applying a template-matching algorithm.

5. The method of claim 1, wherein said tracking the needle tip comprises receiving signals from an electromagnetic sensor attached to the needle.

6. A method of ultrasound needle guidance, the method comprising:
acquiring ultrasound data of a first region-of-interest including a needle;
displaying a first live image in a first viewing pane, the first live image comprising an overview image defined by the first region-of-interest;
tracking a position of a needle tip as the needle is inserted;
establishing a second region-of-interest around the needle tip and automatically adjusting a position of the second region-of-interest to track with the needle tip as the needle is inserted; and
displaying a second live image defined by the second region-of-interest in a second viewing pane at the same time as the first live image, the second live image including the needle tip at a greater level of zoom than the first live image.

7. The method of claim 6, further comprising using both the first live image and the second live image for reference during the process of inserting the needle.

8. The method of claim 6, wherein said tracking the position of the needle comprises implementing an image processing algorithm to identify the needle tip in the first live image.

9. The method of claim 6, wherein said tracking the position of the needle comprises receiving a signal from an electromagnetic sensor attached to the needle.

10. The method of claim 6, wherein the first viewing pane and the second viewing pane are displayed in separate locations on a display device.

11. The method of claim 6, wherein the second viewing pane is superimposed on the first viewing pane on a display device.

12. The method of claim 11, wherein the second viewing pane is positioned where the needle tip would be located in the first viewing pane to provide the effect of magnifying the needle tip.

13. The method of claim 13, further comprising moving the second viewing pane relative to the first viewing pane in order to keep the second viewing pane positioned where the needle tip would be located in the first live image as the needle is inserted.

14. A medical system for providing needle guidance, comprising:
a needle including a needle tip;
a probe, including a plurality of transducer elements;
a display device; and
a processor, wherein the processor is configured to:
control the probe to acquire ultrasound data from a first region-of-interest;
track the needle tip while the needle is moved;
define a second region-of-interest including the needle tip, the second region-of-interest comprising a subset of the first region-of-interest;
adjust a position of the second region-of-interest to track with the needle tip while the needle is moved;
display a first live image of the first region-of-interest on the display device based on the ultrasound data; and
display a second live image of the second region-of-interest on the display device at the same time as the first live image, the second live image including the needle tip and comprising a greater level of zoom than the first live image.

15. The medical system of claim 14, further comprising a magnetic field generator configured to emit a magnetic field, and, wherein the needle includes an electromagnetic sensor sensitive to the magnetic field.

16. The medical system of claim 14, wherein the processor is further configured to track the needle tip by implementing an image processing technique on the first live image.

17. The medical system of claim 16, wherein the processor is further configured to track the needle tip by implementing a template-matching algorithm.

18. The medical system of claim 14, wherein the processor is further configured to display a graphical user interface on the display device, and wherein the graphical user interface is configured to adjust a level of zoom of the second live image.

19. The medical system of claim 14, wherein the processor is further configured to superimpose the second live image over the first live image on the display device.

20. The medical system of claim 19, wherein the processor is further configured to adjust a position of the second live image so that the second live image is positioned where the needle tip would be located needle in the first live image.
